(19) United States
(12) Patent Application Publication
(10) Pub. No.: US 2017/0112790 A1
(43) Pub. Date: Apr. 27, 2017

(54) COMPOSITIONS AND METHODS FOR TREATING ANHIDROSIS

(71) Applicant: Platinum Performance, Inc., Buellton, CA (US)

(72) Inventors: Mark HERTHEL, Los Olivos, CA (US); Meri STRATTON-PHELPS, Fairfield, CA (US)

(21) Appl. No.: 15/359,508

(22) Filed: Nov. 22, 2016

Related U.S. Application Data
(63) Continuation of application No. 13/603,031, filed on Sep. 4, 2012, now abandoned.
(60) Provisional application No. 61/530,702, filed on Sep. 2, 2011.

(51) Int. Cl. A61K 31/198; A61K 31/375; A61K 31/355; A61K 31/14; A61K 31/197; A23L 33/175; A61K 31/4415; A61K 31/506; A61K 31/519; A23K 50/20; A23K 20/142; A23K 20/174; A61K 33/14; A61K 31/4406

(52) U.S. Cl. CPC A61K 31/198; A61K 33/14; A61K 31/375; A61K 31/355; A61K 31/14; A61K 31/197; A61K 31/4406; A61K 31/4415; A61K 31/506; A61K 31/519; A23K 50/20; A23K 20/142; A23K 20/174; A23L 33/175

(57) ABSTRACT

Provided herein are compositions and methods useful for the treatment of anhidrosis in mammals, for example, horses.

### RELATED APPLICATIONS

This application claims the benefit under 35 U.S.C. §119(e) of U.S. Provisional Patent Application No. 61/530,702, filed Sep. 2, 2011, the disclosure of which is incorporated by reference herein in its entirety.

### FIELD

Provided herein are compositions and methods useful for treating or preventing anhidrosis in a mammal in need thereof.

### BACKGROUND

Anhidrosis, or hypohidrosis, is the inability to sweat and can be a debilitating health problem, particularly for horses. Clinically affected horses are unable to dissipate heat by sweating. In some horses, the reduced ability to sweat develops gradually but in other horses, the change is rapid. Anhidrotic horses often show signs of an increased respiratory rate and even respiratory distress as they work to lose heat through their respiratory tract OHubert et al., VCNA, 2002; McEwan Jenkinson et al., Vet. Derm., 2007). A horse's core body temperature may remain elevated, and both their heart rate and blood pressure may also be above normal. Chronically affected horses develop dry, flaky skin as well as thinning of their hair coat. The athletic potential of anhidrotic horses is greatly limited, resulting in the loss of use of the horse unless the horse begins to sweat normally.

The traditional therapy for anhidrotic horses has focused on moving the horse to a cool environment with low humidity. Many horses will resume sweating during the cooler winter months, but horses that are severely affected with anhidrosis may not improve even with appropriate environmental management. While cooling of an anhidrotic horse during the hot summer months can be accomplished in a temperature regulated stall, this management option is not an option for most horse owners leaving few options for managing anhidrosis.

### SUMMARY

In one aspect, provided herein are feed compositions and additives useful for treating or preventing anhidrosis in a mammal in need thereof. Exemplary feed Compositions or additives comprise a nitric oxide enhancer such as L-arginine. The compositions are particularly. useful for treating or preventing anhidrosis in horses.

In another aspect, provided herein are methods of treating or preventing anhidrosis in a mammal in need thereof. The methods comprise the step of administering an amount of a nitric oxide enhancer effective to treat or prevent the anhidrosis in the mammal. The methods are particularly useful for treating or preventing anhidrosis in horses.

### DESCRIPTION

An embodiment of the invention includes a composition comprising a nitric oxide enhancer. The term “nitric oxide enhancer” refers to an agent that increases the amount of nitric oxide. The agent may increase nitric oxide directly (stimulating activity of NO synthase) or indirectly (inhibiting nitric oxide degradation). The nitric oxide enhancer can be any nitric oxide enhancer known to those of skill in the art. Examples include arginine, L-arginine, nitro-L-arginine methyl ester and pterostilbene. A nitric oxide enhancer includes, but is not limited to, L-arginine.

When a horse is unable to sweat, oxidative damage likely increases due to the high environmental heat stress the horse encounters. Therefore natural antioxidants, such as vitamin E and vitamin C, may be helpful in limiting the damaging effects of free radicals and other products of cellular oxidation. An embodiment of the invention includes a composition comprising an antioxidant, such as vitamin A, vitamin C, vitamin E, beta-carotene, or combinations thereof. In certain embodiments, the antioxidants can natural or unnatural. The antioxicants can be obtained by any method known to those of skill including from natural sources, synthetically and semi-synthetically.

An embodiment includes a composition comprising at least one electrolyte supplement. Such electrolytes can be pitassium, sodium, magnesium, calcium, chloride, phosphorus, sulfate, carbonate, and citrate. Such electrolytes can be provided as a salt, for example, potassium chloride, sodium chloride, etc. To prevent the need for additional supplementation, both sodium chloride and potassium chloride can be included to ensure an adequate concentration of these nutrients to prevent a deficiency. The present description includes combinations of the foregoing electrolytes.

An embodiment may also include a composition comprising at least one omega 3 fatty acid, such as α-linoleic acid (ALA), eicosapentaenoic acid (EPA), or docosahexaenoic acid (DHA). The present description includes combinations of the foregoing fatty acids.

An embodiment may also include a composition comprising L-tyrosine.

In certain embodiments, the composition can comprise additional ingredients including vitamins and nutrients. Exemplary vitamins and nutrients include choline, B vitamins, folic acid, and combinations thereof. Useful B vitamins include vitamin B1, vitamin B3, vitamin B5 and vitamin B6.

Each composition provided herein can further include additional ingredients such as carbohydrates and fillers deemed useful by the practitioner of skill in the art.

An embodiment of the invention includes a composition to treat anhidrosis in a mammal. A mammal can include a primate, an equine, or a canine. A mammal can be a human.

Further provided herein are methods of treating or preventing anhidrosis. The methods comprise administering to a mammal in need thereof a composition comprising an amount of a nitric oxide enhancer effective to treat or prevent the anhidrosis. Useful nitric oxide enhancers include arginine, L-arginine, nitro-L-arginine methyl ester and pterostilbene. L-arginine is a particularly useful nitric oxide enhancer.

In humans, there is no current treatment for systemic anhidrosis. Anhidrosis can be diagnosed based on signs and symptoms. Anhidrosis can also be confirmed via testing with a quantitative sudomotor axon reflex test (QSART), silastic sweat imprint, and/or a thermoregulatory sweat test.

In an embodiment, about 4000 mg/day of L-arginine is administered to a horse to treat anhidrosis. In an embodiment, about 4500 mg/day of L-arginine is administered to a horse to treat anhidrosis. In an embodiment, about 4700 mg/day of L-arginine is administered to a horse to treat anhidrosis. In an embodiment, about 5000 mg/day of L-arginine is administered to a horse to treat anhidrosis. In an embodiment, about 6000 mg/day of L-arginine is administered to a horse to treat anhidrosis. In an embodiment, about 7000 mg/day of L-arginine is administered to a horse to treat anhidrosis. In an embodiment, about 8000 mg/day of L-arginine is administered to a horse to treat anhidrosis. In an embodiment, about 9000 mg/day of L-arginine is administered to a horse to treat anhidrosis. In an embodiment, about 10,000 mg/day of L-arginine is administered to a horse to treat anhidrosis. In an embodiment, about 11,000 mg/day of L-arginine is administered to a horse to treat anhidrosis. In an embodiment, about 12,000 nig/day of L-arginine is administered to a horse to treat anhidrosis. In an embodiment, about 12,500 mg/day of L-arginine is administered to a horse to treat anhidrosis. In an embodiment, about 13,000 mg/day of L-arginine is administered to a horse to treat anhidrosis. In an embodiment, about 14,000 mg/day of L-arginine is administered to a horse to treat anhidrosis. In an embodiment, about 14,500 mg/day of L-arginine is administered to a horse to treat anhidrosis. In an embodiment, about 15,000 mg/day of L-arginine is administered to a horse to treat anhidrosis. In an embodiment, about 16,000 mg/day of L-arginine is administered to a horse to treat anhidrosis. In an embodiment, about 17,000 mg/day of L-arginine is administered to a horse to treat anhidrosis. In an embodiment, about 18,000 mg/day of L-arginine is administered to a horse to treat anhidrosis. Such doses can be split into multi-dosing per day regimen. For example, a 14,000 mg/day administration can be split into two 7,000 mg administrations.

In an embodiment, between about 4000 mg/day and about 18,000 mg/day if L-arginine is administered to a horse to treat anhidrosis. In an embodiment, between about 4500 mg/day and about 18,000 mg/day of L-arginine is administered to a horse to treat anhidrosis. In an embodiment, between about 4700 mg/day and about 18,000 mg/day of L-arginine is administered to a horse to treat anhidrosis. In an embodiment, between about 5000 mg/day and about 18,000 mg/day of L-arginine is administered to a horse to treat anhidrosis. In an embodiment, between about 6000 mg/day and about 18,000 mg/day of L-arginine is administered to a horse to treat anhidrosis. In an embodiment, between about 7000 mg/day and about 18,000 mg/day of L-arginine is administered to a horse to treat anhidrosis. In an embodiment, between about 8000 mg/day and about 18,000 mg/day of L-arginine is administered to a horse to treat anhidrosis. In an embodiment, between about 9000 mg/day and about 18,000 mg/day of L-arginine is administered to a horse to treat anhidrosis. In an embodiment, between about 10,000 mg/day and about 18,000 mg/day of L-arginine is administered to a horse to treat anhidrosis. In an embodiment, between about 11,000 mg/day and about 18,000 mg/day of L-arginine is administered to a horse to treat anhidrosis. In an embodiment, between about 12,000 mg/day and about 18,000 mg/day of L-arginine is administered to a horse to treat anhidrosis. In an embodiment, between about 12,500 mg/day and about 18,000 mg/day of L-arginine is administered to a horse to treat anhidrosis. In an embodiment, between about 13,000 mg/day and about 18,000 mg/day of L-arginine is administered to a horse to treat anhidrosis. In an embodiment, between about 14,000 mg/day and about 18,000 mg/day of L-arginine is administered to a horse to treat anhidrosis. Such doses can be split into multi-dosing per day regimen. For example, a 14,000 mg/day administration can be split into two 7,000 mg administrations.

In an embodiment, about 8 mg/kg/day of L-arginine is administered to a mammal to treat anhidrosis In an embodiment, about 9 mg/kg/day of L-arginine is administered to a mammal to treat anhidrosis. In an embodiment, about 10 mg/kg/day of L-arginine is administered to a mammal to treat anhidrosis. In an embodiment, about 12 mg/kg/day of L-arginine is administered to a mammal to treat anhidrosis. In an embodiment, about 14 mg/kg/day of L-arginine is administered to a mammal to treat anhidrosis. In an embodiment, about 16 mg/kg/day of L-arginine is administered to a mammal to treat anhidrosis. In an embodiment, about 18 mg/kg/day of L-arginine is administered to a mammal to treat anhidrosis. In an embodiment, about 20 mg/kg/day of L-arginine is administered to a mammal to treat anhidrosis. In an embodiment, about 22 mg/kg/day of L-arginine is administered to a mammal to treat anhidrosis. In an embodiment, about 24 mg/kg/day of L-arginine is administered to a mammal to treat anhidrosis. In an embodiment, about 25 mg/kg/day of L-arginine is administered to a mammal to treat anhidrosis. In an embodiment, about 26 mg/kg/day of L-arginine is administered to a mammal to treat anhidrosis. In an embodiment, about 28 mg/kg/day of L-arginine is administered to a mammal to treat anhidrosis. In an embodiment, about 29 mg/kg/day of L-arginine is administered to a mammal to treat anhidrosis. In an embodiment, about 30 mg/kg/day of L-arginine is administered to a mammal to treat anhidrosis. In an embodiment, about 32 mg/kg/day of L-arginine is administered to a mammal to treat anhidrosis. In an embodiment, about 34 mg/kg/day of L-arginine is administered to a mammal to treat anhidrosis. In an embodiment, about 18,000 mg/kg/day of L-arginine is administered to a mammal to treat anhidrosis. Such doses can be split into multi-dosing per day regimen. For example, a 28 mg/kg/day administration can be split into two 14 mg/kg/day administrations.

In an embodiment, between about 8 mg/kg/day and about 36 mg/kg/day of L-arginine is administered to a mammal to treat anhidrosis. In an embodiment, between about 9 mg/kg/day and about 36 mg/kg/day of L-arginine is administered to a mammal to treat anhidrosis. In an embodiment, between about 10 mg/kg/day and about 36 mg/kg/day of L-arginine is administered to a mammal to treat anhidrosis. On an embodiment, between about 12 mg/kg/day and about 36 mg/kg/day of L-arginine is administered to a mammal to treat anhidrosis. In an embodiment, between about 14 mg/kg/day and about 36 mg/kg/day of L-arginine is administered to a mammal to treat anhidrosis. In an embodiment, between about 16 mg/kg/day and about 36 mg/kg/day of L-arginine is administered to a mammal to treat anhidrosis. In an embodiment, between about 18 mg/kg/day and about 36 mg/kg/day of L-arginine is administered to a mammal to treat anhidrosis. In an embodiment, between about 20 mg/kg/day and about 36 mg/kg/day of L-arginine is administered to a mammal to treat anhidrosis. In an embodiment, between about 22 mg/kg/day and about 36 mg/kg/day of L-arginine is administered to a mammal to treat anhidrosis. In an embodiment, between about 24 mg/kg/day and about 36 mg/kg/day of L-arginine is administered to a mammal to treat anhidrosis. In an embodiment, between about 25 mg/kg/day and about 36 mg/kg/day of L-arginine is administered to a mammal to treat anhidrosis. In an embodiment, between about 26 mg/kg/day and about 36 mg/kg/day of L-arginine is administered to a mammal to treat anhidrosis. In an embodiment, between about 28 mg/kg/day and about 36 mg/kg/day of L-arginine is administered to a mammal to treat anhidrosis. Such doses can be split into multi-dosing per day regimen. For example, a 28 mg/kg/day administration can be split into two 7,000 mg/kg/day administrations.

In an embodiment, about 8 mg/kg/day of L-arginine is administered to a human to treat anhidrosis. In an embodiment, about 9 mg/kg/day of L-arginine is administered to a human to treat anhidrosis. In an embodiment, about 10 mg/kg/day of L-arginine is administered to a human to treat anhidrosis. In an embodiment, about 12 mg/kg/day of L-arginine is administered to a human to treat anhidrosis. In an embodiment, about 14 mg/kg/day of L-arginine is administered to a human to treat anhidrosis. In an embodiment, about 16 mg/kg/day of L-arginine is administered to a human to treat anhidrosis. In an embodiment, about 18 mg/kg/day of L-arginine is administered to a human to treat anhidrosis. In an embodiment, about 20 mg/kg/day of L-arginine is administered to a human to treat anhidrosis. In an embodiment, about 22 mg/kg/day of L-arginine is administered to a human to treat anhidrosis. In an embodiment, about 24 mg/kg/day of L-arginine is administered to a human to treat anhidrosis. In an embodiment, about 25 mg/kg/day of L-arginine is administered to a human to treat anhidrosis. In an embodiment, about 26 mg/kg/day of L-arginine is administered to a human to treat anhidrosis. In an embodiment, about 28 mg/kg/day of L-arginine is administered to a human to treat anhidrosis. In an embodiment, about 29 mg/kg/day of L-arginine is administered to a human to treat anhidrosis. In an embodiment, about 30 mg/kg/day of L-arginine is administered to a human to treat anhidrosis. In an embodiment, about 32 mg/kg/day of L-arginine is administered to a human to treat anhidrosis. In an embodiment, about 34 mg/kg/day of L-arginine is administered to a human to treat anhidrosis. In an embodiment, about 18,000 mg/kg/day of L-arginine is administered to a human to treat anhidrosis. Such doses can be split into multi-dosing per day regimen. For example, a 28 mg/kg/day administration can be split into two 14 mg/kg/day administrations.

In an embodiment, between about 8 mg/kg/day and about 36 mg/kg/day of L-arginine is administered to a human to treat anhidrosis. In an embodiment, between about 9 mg/kg/day and about 36 mg/kg/day of L-arginine is administered to a human to treat anhidrosis. In an embodiment, between about 10 mg/kg/day and about 36 mg/kg/day of L-arginine is administered to a human to treat anhidrosis. In an embodiment, between about 12 mg/kg/day and about 36 mg/kg/day of L-arginine is administered to a human to treat anhidrosis. In an embodiment, between about 14 mg/kg/day and about 36 mg/kg/day of L-arginine is administered to a human to treat anhidrosis. In an embodiment, between about 16 mg/kg/day and about 36 mg/kg/day of L-arginine is administered to a human to treat anhidrosis. In an embodiment, between about 18 mg/kg/day and about 36 mg/kg/day of L-arginine is administered to a human to treat anhidrosis. In an embodiment, between about 20 mg/kg/day and about 36 mg/kg/day of L-arginine is administered to a human to treat anhidrosis. In an embodiment, between about 22 mg/kg/day and about 36 mg/kg/day of L-arginine is administered to a human to treat anhidrosis. In an embodiment, between about 24 mg/kg/day and about 36 mg/kg/day of L-arginine is administered to a human to treat anhidrosis. In an embodiment, between about 25 mg/kg/day and about 36 mg/kg/day of L-arginine is administered to a human to treat anhidrosis. In an embodiment, between about 26 mg/kg/day and about 36 mg/kg/day of L-arginine is administered to a human to treat anhidrosis. In an embodiment, between about 28 mg/kg/day and about 36 mg/kg/day of L-arginine is administered to a human to treat anhidrosis. Such doses can be split into multi-dosing per day regimen. For example, a 28 mg/kg/day administration can be split into two 7,000 mg/kg/day administrations.

As used herein, the term “about” has the meaning known to those of skill in the art. When applied to a percent amount, the term about indicates that percent amount ±0.5%. When applied to an absolute amount, the term about indicates that amount ±2.5%. As used herein, a “substantial” amount refers to an amount sufficient to treat or prevent anhidrosis in a mammal in need thereof. In certain embodiments, the term “substantial” indicates at least 20% of the composition is the indicated ingredient. In certain embodiments, the term “substantial” indicates that a single serving of the composition includes at least about 8,000 mg of the indicated ingredient.

In the compositions provided herein, the active and inactive ingredients can be prepared by any process apparent to those of skill in the art. In particular embodiments, the ingredients are obtained from commercial sources.

### EXAMPLES

As used herein, the symbols and conventions used in these processes, schemes and examples, regardless of whether a particular abbreviation is specifically defined, are consistent with those used in the contemporary scientific literature.

### Example 1

An embodiment is the following composition formulated for oral administration to a horse twice a day.

TABLE 1

| | Formula | Label Claim per | |
|---|---|---|---|
| Ingredient | per Serving (mg) | Serving (mg) | Percent |
| Potassium Chloride | 6250.00 | 6250.00 | 30.49% |
| NaCl | 5000.00 | 5000.00 | 24.39% |
| L-Arginine | 4750.00 | 4750.00 | 23.17% |
| Vitamin C | 2500.00 | 2500.00 | 12.20% |
| Vitamin E 630 | 500.00 | 500.00 | 2.44% |
| L-Tyrosine | 720.00 | 720.00 | 3.51% |
| Choline Bitartrate | 375.00 | 375.00 | 1.83% |
| Vitamin B5 - Calcium | 175.00 | 175.00 | 0.85% |
| Pantothenate | | | |
| Vitamin B3 - Niacin | 100.00 | 100.00 | 0.49% |
| Vitamin B6 - | 100.00 | 100.00 | 0.49% |
| Pyridoxine HCl | | | |
| Vitamin B1 - Thiamin | 25.00 | 25.00 | 0.12% |
| Folic Acid | 5.00 | 5.00 | 0.02% |
| Totals | 20,500.00 | 20,500.00 | 100.00% |

### Example 2

Horses with anhidrosis were treated with the formulation provided in Example 1 twice per day (Table 2).

TABLE 2

| Horse | Stopped Sweating | Sweating at 1 month | Sweating at 2 months |
|---|---|---|---|
| 1 | Chronic | No | No |
| 2 | | Yes | |
| 3 | Jun. 20, 2011 | No | Yes |
| 4 | Jun. 1, 2011 | No | Better but not 100% |
| 5 | Jun. 5, 2011 | No | Better but not 100% |
| 6 | May 2011 | No | |
| 7 | May 2011 | No | |
| 8 | May 2011 | No | |
| 9 | May 2011 | No | |
| 10 | May 2011 | No | |
| 11 | 2008 | Yes | |
| 12 | May 2011 | Yes | |
| 13 | May 2011 | No | No |
| 14 | 2006 | Yes | No |
| 15 | Summer 2010 | No | |

Out of 15 horses administered the composition described in Example 1, four (4) horses resumed sweating by at least one month after administration started. At two months, another horse was fully sweating again, and two horses showed improved, but not complete sweating. Overall, administration of the composition of Example 1 restored or improved the sweating in 7 of 15 horses.

### Example 3

An embodiment o composition formulated for oral administration to a horse twice a day.

TABLE 3

| | | | Label Claim per |
|---|---|---|---|
| Ingredients | Percent | Formula (mg) | Serving (mg) |
| L-Arginine | 34.01% | 7617.963 | 7500.0 |
| Potassium Chloride | 27.96% | 6262.525 | 6250.0 |
| NaCl | 22.51% | 5041.575 | 5000.0 |
| Vitamin C | 6.19% | 1387.626 | 1250.0 |
| L-Tyrosine | 3.28% | 734.473 | 720.0 |
| Vitamin E 630 | 2.25% | 505.000 | 500.0 |
| Choline Bitartrate | 1.77% | 397.538 | 375.0 |
| Vitamin B5 - Calcium | 0.88% | 196.071 | 175.0 |
| Pantothenate | | | |
| Vitamin B3 - Niacin | 0.50% | 111.523 | 100.0 |
| Vitamin B6 - Pyridoxine | 0.50% | 112.041 | 100.0 |
| HCl | | | |
| Vitamin B1 - Thiamin | 0.13% | 28.010 | 25.0 |
| Folic Acid | 0.03% | 5.655 | 5.0 |
| Totals | 100.00% | 22,400.000 | 22,000.0 |

### Example 4

The embodiment of the compositions provided in Example 3 was administered to horses having one or more symptoms of anhidrosis. As detailed in the following table, some of the horses had no prior treatments for anhidrosis, and some of the horses had one or more than one prior treatments for anhidrosis. The results of the administrations are summarized in Table 4. These results were reported by voluntarily by individuals that had administered the composition and observed the results.

TABLE 4

| | | | | | | | | Results |
|---|---|---|---|---|---|---|---|---|
| | | | | | | | | from |
| | | | | | Results from | Treatment | | Treatment |
| | | Condition | | Prior | Prior | with | | with |
| Horse | Condition | Duration | | Treatment | Treatment | Example 3 | | Example 3 |
| 1 | Anhidrosis | | | | | 10 | days | Sweating |
| 2 | Poor | | | | | Yes | | Sweating |
| | sweating | | | | | | | better |
| 3 | Poor | | | One AC, Leg | Some | Yes | | Sweating |
| | sweating | | | Magic | improvement, | | | |
| | | | | | short duration | | | |
| 4 | Non- | | | ProSweat, | No Success | 3 | wks. | Sweating |
| | sweater | | | Make 'Em | | | | |
| | | | | Sweat, One | | | | |
| | | | | AC | | | | |
| 5 | Non- | Chronic | | | | Yes | | Sweating |
| | sweater | | | | | | | better than |
| | | | | | | | | ever |
| 6 | | | | | | 2 | wks | Marked |
| | | | | | | | | improvement |
| 7 | | | | One AC, | Minimal | Yes | | Better |
| | | | | True Sweat | success | | | sweating |
| 8 | Anhidrosis | 4-5 | mos. | None | 4-5 months | 3-4 | wks. | Results seen |
| | | | | | anhidrosis | | | |
| 9 | Anhidrosis | 1 | yr. | One AC | Anhidrosis 1 | Yes | | Sweating, |
| | | | | | year | | | respiration |
| | | | | | | | | improved |
| 10 | Anhidrosis | 4 | yrs. | One AC | 3 months | 2 | wks. | Results seen |
| | | | | | some | | | |
| | | | | | sweating | | | |
| 11 | Anhidrosis | 10 | yrs. | One AC | No success | 1 | wk. | Results seen |
| 12 | Anhidrosis | 6 | yrs. | One AC | Success | Immediate | | Success |
| 13 | Anhidrosis | 1 | yr. | One AC, | No | 2-3 | wks. | Results |
| | | 2 | mos. | acupuncture, | | | | observed |
| | | | | Sweat | | | | |
| | | | | Activator | | | | |
| 14 | Anhidrosis | 3 | yrs. | One AC | Some | 1 | wk. | Results |
| | | | | | | | | observed |
| 15 | Anhidrosis | Year- | | One AC, | No | 1 | mo. | Results |
| | | round | | acupuncture, | | | | |
| | | | | herbs | | | | |
| 16 | Anhidrosis | 2½ | yrs. | One AC | 3 months, no | 45 | days | Results |
| 17 | Anhidrosis | 1 | yr. | One AC | 3 months, | Yes | | Success |
| | | | | | minimal | | | |
| | | | | | success | | | |
| 18 | Anhidrosis | 5 | mos. | Acupuncture | 1-3 months, | One | mo. | Response |
| | | | | | no success | | | |
| 19 | Anhidrosis | Summers | | One AC | 3 months, no | Yes | | Sweating |
| | | | | | success | | | |
| 20 | Anhidrosis | 3 | | One AC | 1-3 months, | Yes | | Sweating |
| | | summers | | | success first | | | normally |
| | | | | | summer only, | | | |
| | | | | | no success | | | |
| | | | | | subsequent | | | |
| | | | | | summers | | | |
| 21 | Anhidrosis, | 2 | mos. | One AC, | No success | 4 | wks. | Sweating |
| | two | | | electrolytes | | | | |
| | months | | | | | | | |
| 22 | | | | | | Yes | | Success |
| 23 | | | | One AC, Let | No success | Yes | | Sweating |
| | | | | 'Em Sweat, | | | | |
| | | | | Salt, Beer | | | | |
| 24 | | | | | | 5 | days | Sweating |
| 25 | Anhidrosis | 1 | yr. | Acupuncture, | Little or no | 23 | days | Sweating |
| | | | | herbs, One | sweating | | | |
| | | | | AC, beer, | | | | |
| | | | | True Sweat | | | | |
| 26 | Anhidrosis | 1 | mo. | | | 1 | mo. | Sweating |
| 27 | Anhidrosis | 1 | yr. | True Sweat | No sweating | 2 | days | Sweating, |
| | | | | | behind saddle | | | back and |
| | | | | | | | | hindquarters |

| | | | | | |
|---|---|---|---|---|---|
| 28 | Anhidrosis | Chronic | 7 | days | Sweating |

All publications and patent, applications cited in this specification are herein incorporated by reference as if each individual publication or patent application were specifically and individually indicated to be incorporated by reference. While the claimed subject matter has been described in terms of various embodiments, the skilled artisan will appreciate that various modifications, substitutions, omissions, and changes may be made without departing from the spirit thereof. Accordingly, it is intended that the scope of the subject matter limited solely by the scope of the following claims, including equivalents thereof.

1.-13. (canceled)

14. A method of treating or preventing anhidrosis comprising the step of administering at least about 8000 mg/day L-arginine to a mammal in need thereof.

15. The method of claim 14 comprising administering between about 8000 mg/day and 18,000 mg/day L-arginine to a mammal in need thereof.

16. The method of claim 15 wherein said mammal is a horse.

17. A method of treating or preventing anhidrosis comprising the step of administering a feed composition or additive to a mammal in need thereof; wherein the feed composition or additive comprises a substantial amount of a nitric oxide enhancer for the treatment or prevention of anhidrosis in mammals.

18. The method of claim 17 wherein said mammal is a horse.

19. A method of treating or preventing anhidrosis comprising the step of administering a feed composition or additive to a mammal in need thereof; wherein the feed composition or additive comprises about 30.5% potassium chloride, about 24.5% sodium chloride, about 23% L-arginine, about 12.2% vitamin C, about 2.4% vitamin E 360, about 3.5% L-tyrosine, about 1.8% choline bitartrate, about 0.8% vitamin B5, about 0.5% vitamin B3, about 0.5% vitamin B6, about 0.1% vitamin B1 and about 0.02% folic acid.

20. The method of claim 19 wherein said mammal is a horse.
